(19) United States
(12) Patent Application Publication
(10) Pub. No.: US 2023/0274830 A1
(43) Pub. Date: Aug. 31, 2023

(54) METHOD FOR PERCEPTIVE TRAITS BASED SEMANTIC FACE IMAGE MANIPULATION AND AESTHETIC TREATMENT RECOMMENDATION

(71) Applicant: RealFaceValue B.V., Rotterdam (NL)

(72) Inventors: Jacques van der Meulen, Rotterdam (NL); Ekin Gedik, Delft (NL); Berno Bucker, Amsterdam (NL)

(21) Appl. No.: 17/681,224

(22) Filed: Feb. 25, 2022

(51) Int. Cl. G16H 50/20; G06V 40/16; G06V 10/82; G16H 20/40; G06N 3/04

(52) U.S. Cl. CPC G16H 50/20; G06V 40/167; G06V 10/82; G16H 20/40; G06N 3/0454

(57) ABSTRACT

One aspect is a method for generating an image of a human face expected to have at least one changed perceptive trait. Such method includes encoding an image of a human face into a vector to generate an input image vector. A scaled direction vector is applied to the input image vector to generate a modified input image vector. The modified input image vector is converted into a morphed image of the actual human face. The scaled direction vector is determined using an artificial neural network trained to associate human faces to with ratings of perceptive traits, to determine a unit direction vector. The unit direction vector is based on at least one chosen perceptive trait. An amplitude for scaling the unit direction vector is determined by a limit on change in facial features obtainable by known treatments.

### CROSS REFERENCE TO RELATED APPLICATIONS

Not Applicable.

### STATEMENT REGARDING FEDERALLY SPONSORED RESEARCH OR DEVELOPMENT

Not Applicable

### NAMES OF THE PARTIES TO A JOINT RESEARCH AGREEMENT

Not Applicable.

### BACKGROUND

This disclosure relates to the field of processing images of a human anatomical feature, such as the face, to project expected appearance of the face as a result of contemplated treatment. Such treatments may include cosmetic treatment, surgical treatment or medical treatment such as injection of fillers and other agents, although the disclosure is not limited to medical treatments. More specifically, the disclosure relates to methods for creating an expected image based on machine learning processing of actual changes in appearance resulting from facial treatments on actual persons. The disclosure further relates to methods for recommending a suitable treatment to obtain an improvement in one or more perceptive traits associated with the human face.

Cosmetic treatments, e.g., medical cosmetic treatments, surgical treatments and injections (for example, but not limited to Botox and fillers), are undertaken to modify appearance of human features, such as the face. While physicians, in particular plastic surgeons, have detailed knowledge of how to perform such procedures, and have informed understanding of how such procedures will alter the appearance of human features, it is difficult to convey to the person what may be reasonably expected as a result of such procedures. Further, the physician may have the ability to choose the amount by which features are changed with reference to the intensity of a procedure or the product used, but may still be unable to convey to the person how variations in the amount of feature change will affect the overall appearance when the treatments are completed.

Appearance of a human face can create perceptions in human observers which can be quantified. U.S. Pat. No. 11,151,362 issued to Velthuis et al. discloses a system that receives as input an image of a face of a person and determines landmarks on the face, indicating properties of predetermined anatomical portions of the face, by analyzing the image using a set of image processing and deep learning algorithms. The system compares the landmarks to a model generated based on faces scored for a plurality of perceptive traits through scientifically validated surveys by people. The system determines, using the model, a score for each of the plurality of perceptive traits for the face based on the comparison. The system determines, using the model, a first impression for the face collectively based on the scores for all of the perceptive traits determined by the model for the face. The system provides an output comprising the first impression and the scores for the perceptive traits determined by the model for the face.

In addition to the system disclosed in the '362 patent, other methods known in the art provide ways to alter an input image to display expected appearance of the human face after one or more such treatments. What is needed is a tool to enable suggestion of suitable treatments to improve observer rating of one or more perceptive traits of a human face.

### SUMMARY

One aspect of the present disclosure is a method for generating an image of a human face expected to have at least one changed perceptive trait. A method according to this aspect of the present disclosure includes encoding an image of a human face into a vector to generate an input image vector. A scaled direction vector is applied to the input image vector to generate a modified input image vector. The modified input image vector is converted into a morphed image of the actual human face. The scaled direction vector is determined using an artificial neural network trained to associate human faces to with ratings of perceptive traits, to determine a unit direction vector. The unit direction vector is based on at least one chosen perceptive trait. An amplitude for scaling the unit direction vector is determined by a limit on change in facial features obtainable by any available treatment or facial adjustment.

In some embodiments, the unit direction vector is determined by selecting, from among a plurality of human face rating images, ones of the human face rating images rated highest and ones of the human face rating images rated lowest, the rating performed by the artificial neural network for the at least one chosen perceptive trait, and determining a direction of the unit direction vector from vector representations of the selected highest-rated images and lowest-rated images.

In some embodiments, the human face rating images comprise randomly generated artificial human face images, the randomly generated artificial human face images generated by a generative adversarial network.

Some embodiments further comprise using the input image and the morphed image to automatically generate at least one recommended treatment to cause the human face from which the input image was made to most closely match the morphed image.

In some embodiments, the automatically generating the at least one recommended treatment comprises entering the input image and the morphed image into an artificial neural network trained for change detection to detect changed specific regions in the input image; comparing the detected changed regions to face region masks known to be affected by specific treatments; and if an intersection of at least one of the detected changed regions with at least one of the face region masks is greater than a predefined threshold, the specific treatment is added to a suggested treatment list.

In some embodiments, the changed regions are detected by calculating a difference image between the image of an actual human face and the morphed image.

In some embodiments, the changed regions are detected by entering the image of an actual human face and the morphed image into an artificial neural network trained on change detection.

In some embodiments, the automatically generating the at least one recommended treatment comprises entering the input image and the morphed image into an artificial neural network trained by entering before treatment images and after treatment images of actual human faces having undergone at least one treatment.

In some embodiments, the automatically generating the at least one recommended treatment comprises comparing a direction vector of the input image with reference to the morphed image with direction vectors of each of a plurality of treatments and selecting one of the plurality of treatments that most closely matches the direction vector of the input image with reference to the morphed image.

In some embodiments, the encoded image is of an actual human face.

A computer program according to another aspect of the present disclosure is stored in a non-transitory computer readable medium. The program comprises logic operable to cause a programmable computer to perform acts comprising the following. An image of a human face is encoded into a vector to generate an input image vector. A scaled direction vector is applied to the input image vector to generate a modified input image vector. The modified input image vector is converted into a morphed image of the actual human face. The scaled direction vector is determined by training a first artificial neural network to determine changes in appearance of human face images corresponding to changes in perceptive traits to determine a direction vector based on at least one chosen perceptive trait, and an amplitude for scaling the direction vector is determined by a limit on change in facial features obtainable by known treatments.

In some embodiments, the unit direction vector is determined by selecting, from among a plurality of human face rating images, ones of the human face rating images rated highest and ones of the human face rating images rated lowest, the rating performed by the artificial neural network for the at least one chosen perceptive trait, and determining a direction of the unit direction vector from vector representations of the selected highest-rated images and lowest-rated images.

In some embodiments, the human face rating images comprise randomly generated artificial human face images, the randomly generated artificial human face images generated by a generative adversarial network.

Some embodiments further comprise using the input image and the morphed image to automatically generate at least one recommended treatment to cause the human face from which the input image was made to most closely match the morphed image.

In some embodiments, the automatically generating the at least one recommended treatment comprises entering the input image and the morphed image into an artificial neural network trained for change detection to detect changed specific regions in the input image; comparing the detected changed regions to face region masks known to be affected by specific treatments; and if an intersection of at least one of the detected changed regions with at least one of the face region masks is greater than a predefined threshold, the specific treatment is added to a suggested treatment list.

In some embodiments, the changed regions are detected by calculating a difference image between the image of an actual human face and the morphed image.

In some embodiments, the changed regions are detected by entering the image of an actual human face and the morphed image into an artificial neural network trained on change detection.

In some embodiments, the automatically generating the at least one recommended treatment comprises entering the input image and the morphed image into an artificial neural network trained by entering before treatment images and after treatment images of actual human faces having undergone at least one treatment.

In some embodiments, the automatically generating the at least one recommended treatment comprises comparing a direction vector of the input image with reference to the morphed image with direction vectors of each of a plurality of treatments and selecting one of the plurality of treatments that most closely matches the direction vector of the input image with reference to the morphed image.

In some embodiments, the encoded image is of an actual human face.

Other aspects and possible advantages will be apparent from the description and claims that follow.

### BRIEF DESCRIPTION OF THE DRAWINGS

FIG. 1 shows a flow chart of a process for training a neural network to determine direction vectors for morphing human face images so as to improve expected rating of one or more chosen perceptive traits.

FIG. 2 shows a flow chart of a process for morphing an input image of an actual human face in a manner that will result in improvement to one or more chosen perceptive traits.

FIG. 3 shows a flow chart of one example embodiment of a treatment recommendation method.

FIG. 4 shows a flow chart of an example embodiment of a treatment recommendation method.

FIG. 5 shows a schematic diagram of an example computer system that may be used in some embodiments.

### DETAILED DESCRIPTION

This disclosure explains a procedure to generate realistically modified human face images that are expected to score differently (higher or lower) in one or more selected perceptive traits and a recommender that suggests treatments, e.g., surgical, cosmetic and other medical treatments which are expected to result in changes to a human face, for example, an actual human's face, which would result in change, e.g., improvement, to the one or more perceptive traits when the treated person is observed by other people. As used in this disclosure, the term “image” is not limited to still images. Moving images, e.g., video, is entirely within the scope of the present disclosure.

The procedure is based on the use of neural networks such as Convolutional Neural Networks (CNNs), a face image training set with corresponding perceptive trait labels, and Generative Adversarial Networks (GANs).

In an example embodiment of a method, a GAN that is trained on face images is used. The GAN is pre-trained to generate random images of human faces that are realistic in appearance. The GAN generated images are used in the disclosed method to identify and rate one or more perceptive traits and to determine a latent space direction and vector amplitude to morph the face image, e.g., the image of a natural human whose image is used as input to improve the one or more perceptive traits. The method then determines one or more treatments that may result in changes in the person's face that will result in the improved one or more perceptive traits. As used in this disclosure, the term “treatment” is intended to include the action of any agent, structural modification or manipulation, whether permanent or temporary, including, without limitation, surgical, cosmetic and other medical treatments, manipulations, changes in color and Fitzpatrick skin type, which are expected to result in changes to the appearance of a human face. Accordingly, the specific examples of treatments provided herein are intended only to provide examples of possible embodiments and not to in any way limit the scope of the present disclosure.

A method or procedure according to the present disclosure may be implemented on a computer or computer system, to be explained further below. A non-transitory computer readable medium may have stored thereon instructions or logic operable to program a programmable computer or computer system to perform a method or procedure according to the present disclosure.

An example embodiment of a method or procedure according to the present disclosure may begin by training a neural network, such as a convolutional neural network (CNN) to automatically rate one or more perceptive traits of an image, e.g., of a human face. In the present example embodiment, the CNN may be trained using results of surveys made by human observers who are shown human face images and indicate their subjective rating of one or more perceptive traits. An example embodiment of a procedure to train the CNN is described in U.S. Pat. No. 11,151,362 issued to Velthuis et al., incorporated herein by reference. The present example embodiment is described in terms of training a CNN, however, a pre-trained CNN may be used if such pre-trained CNN includes automated rating of the specific perceptive traits to be improved using a procedure as disclosed herein. The trained CNN may be based, for example, on a validated database comprising a compilation of numerous faces of people that are rated or scored by other people with diverse backgrounds, socio-economic statuses, age groups, etc. (i.e., people with a wide spread regarding their demographics). The faces are scored based on a plurality of perceptive traits. The perceptive traits include, for example and without limitation, personality attributions (e.g., dominant, attractive, kind, etc.) and socio-economic factors (e.g., rich, fashionable, etc.) that are associated with a person's face upon forming a first impression about the person's face. Non-limiting examples of the perceptive traits include attractiveness, competence, dominance, intelligence, warmth, and trustworthiness.

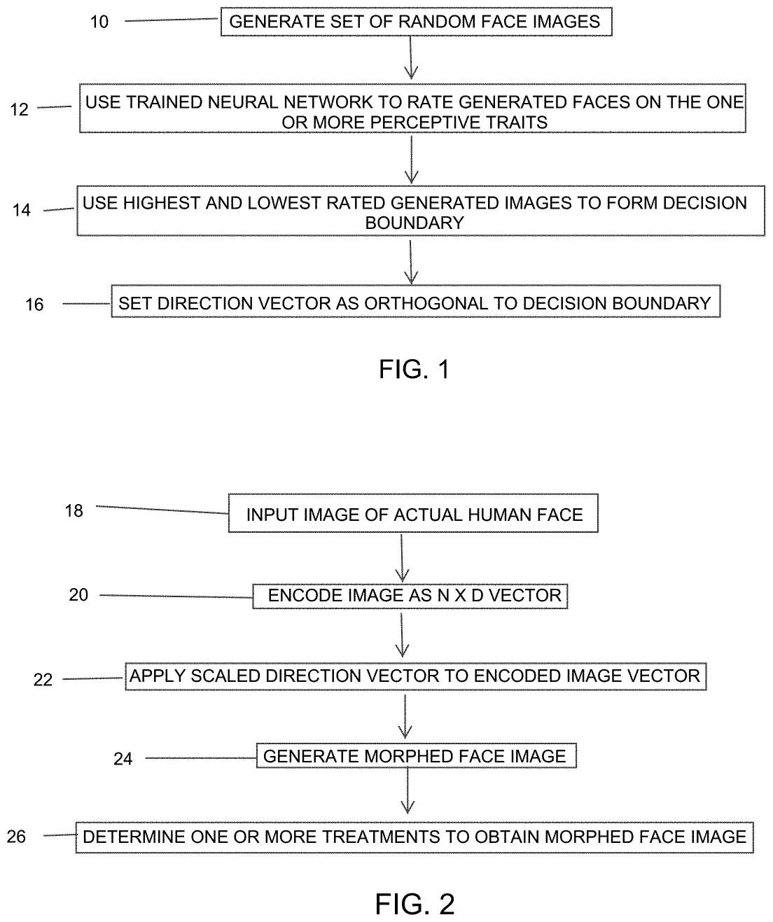

Referring to FIG. 1, and as explained above, a trained GAN may be used, at 10, to generate a set of face images at random. The set of face images may number on the order of 100,000 to 1,000,000 individual images each of a different human face generated by the GAN, although the exact number of images generated is not intended to limit the scope of the present disclosure.

At 12, each image in the set of random face images generated by the GAN may then be rated by the trained CNN on any one or more perceptive traits. At 14, from among the rated images, a predetermined number of such rated images may be selected as representative of the highest (numerical) values and the lowest values of the one or more selected perceptive traits. N x D vectors for the highly rated images and the lowly rated images may then be used as input to train a binary classifier. At 14, a decision boundary is determined, wherein the N x D vectors of the highly rated images are disposed on one side of the decision boundary, and the N x D vectors of the lowly rated images are located on the other side of the decision boundary. A vector normal to the decision boundary, determined at 16, represents a direction to apply to an N×D dimension encoded image vector of an image of an actual human face (the “input image”) in order to improve the one or more selected perceptive traits.

Having determined a direction for the N×D image vector, the described procedure next determines an amplitude related to the one or more selected perceptive traits. In some embodiments, the amplitude may be determined by iteratively changing the amplitude, vector adding it to the N x D vector representation of the image vector, and sending the manipulated image vector representation to the trained GAN to generate a manipulated image. The foregoing may be repeated with incrementally changed amplitude until a final value of the amplitude is reached.

The amplitude corresponds to the strength of the change made to the appearance of the face. The amplitude determination process is implemented to identify the maximum amplitude that results in facial changes which still preserve the identity of the face, and where such changes are obtainable by aesthetic, cosmetic or other types of treatments. The identified value acts as a natural (social) limit to facial changes. It is understood by those skilled in the art that at some amount of change, the morphed face appearance is outside the range of subjectively acceptable appearances of the human face when observed by actual humans.

In some embodiments, the final value of the amplitude may be limited by entering into the computer or computer system a hard-coded upper limit for the amplitude. In some embodiments, the hard coded upper limit may be obtained as follows:

In some embodiments, change in landmarks may be used: Changes in the landmarks on the face that are associated with regions of the face that the treatment is affecting are evaluated both before and after a treatment is performed using the input image and the manipulated image. When a difference or ratio between selected landmarks reaches a predefined limit further changes in landmarks are terminated and the then-current amplitude value is used. If the predefined limit has not been reached, the landmarks may be changed, and the process returns to the act of determining changes in the landmarks. The foregoing ratio or difference may be understood as being related to distances between various specific points on the anatomical feature, e.g., the human face. It is understood by medical practitioners that certain distances or ratios of distances between certain anatomical features are within empirically determined limits. Such empirically determined limits may be used as the limit for the amplitude determination.

In some embodiments, when changes in the color and the texture of the affected region reach a predefined limit, the process is stopped and the then-current amplitude value is used as the final value of amplitude. Predefined limits for landmarks and color may be obtained for example using before treatment and after treatment images of actual human anatomical features.

In some embodiments, constraints on the value of the amplitude may be manually defined by a subject matter expert after viewing multiple manipulated face images with various amplitude values.

In some embodiments, additional constraints on the value of the amplitude may automatically defined by a CNN that is trained in facial recognition.

When a direction and amplitude for adjusting an input image vector are determined, the result is a scaled direction vector. A morphed image may be generated by vector adding the scaled direction vector to a vector representation of an input image. A scaled direction vector may be determined for any one or more chosen perceptive traits, such that application of the scaled direction vector to an input image of a human face will generated a morphed image that may be expected to have improvement in the one or more selected perceptive traits.

Referring to FIG. 2, an image of an actual human face, at 18, may be morphed using a GAN trained as explained with reference to FIG. 1. The input image may be encoded into a N x D vector to form an input image vector at 20. The scaled direction vector, at 22, may be applied to the input image vector. At 24, a morphed image may be generated using the input image vector and the scaled direction vector.

At 26, the morphed image may then be used as input by the process to generate one or more treatment suggestions, which suggested treatments when applied to the face of the human used to generate the input image, will most closely result in the morphed image.

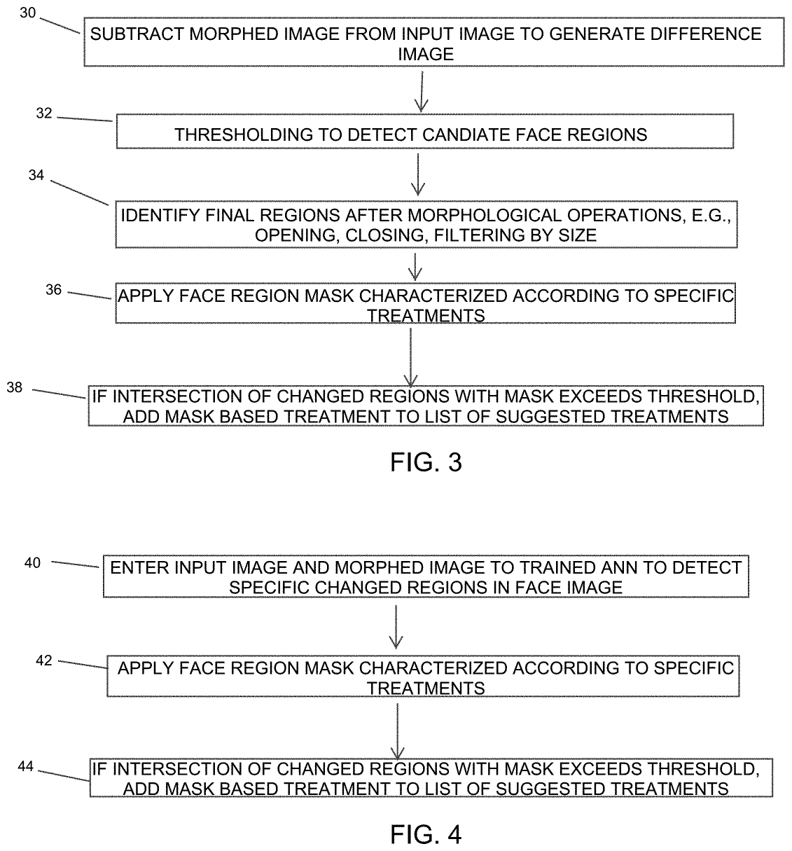

In some embodiments, and referring to FIG. 3, changed regions in the human face may be detected by an unsupervised or a supervised change detection method: In an unsupervised example embodiment of a change detection method, a difference image, e.g., a grayscale difference image, may calculated, at 30, by subtracting the morphed image from the input image, automatic thresholding is applied to the difference image, at 32, to detect candidate regions within the image, and at 34, which is an optional action and may be included in some embodiments, final regions are identified after morphological operations on the candidate regions such as opening, closing and filtering by size.

Regions identified as detected changed regions, at 36 are then compared to face region masks that are known to be affected by specific treatments. If the percentage of the intersection of the changed region with the region mask is higher than a predefined threshold, as shown at 38, the treatment that is known to affect the region mask is added to a suggested treatment list. The suggested treatment(s) (e.g., in a list for multiple recommendations) may be ordered with respect to intersection percentages and then presented to the user.

In a supervised example embodiment of a change detection method, and referring to FIG. 4, an artificial neural network trained for change detection is used to detect changed specific regions in the face image. This is shown in FIG. 4 at 40.

Regions identified in detected changed regions, at 42 are then compared to face region masks that are known to be affected by specific treatments. If the percentage of the intersection of the changed region with the region mask is higher than a predefined threshold, as shown at 44, the treatment that is known to affect the region mask is added to a suggested treatment list. The suggested treatment(s) (e.g., in a list for multiple recommendations) may be ordered with respect to intersection percentages and then presented to the user.

In one embodiment, a neural network such as a CNN may be trained on a data set comprising pre and post-treatment images of actual human faces having undergone specific treatments. The CNN when trained may act as a classifier to that can determine, between two images of a human face, whether and which treatments have been performed on such human face. To suggest treatment(s) for a person contemplating treatment(s) to obtain appearance of the face that corresponds to the image having higher value(s) of the chosen perceptive trait(s), the original face image and the morphed face image are input to the trained CNN. The trained CNN then can output treatment suggestions to cause the original image to most closely approximate the morphed image.

In one embodiment, identified perceptive trait direction, that is, the vector direction to cause one image to be changed in a specific way to change a chosen perceptive trait, can be compared to previously identified directions for various treatments. Comparison can be done using the exact identified direction or using extracted components of the identified direction. If a treatment direction is found to be similar to the identified (perceptive) direction or one of its components, the treatment can be suggested to the user.

Although the present disclosure is made with reference to increasing the value of at least one perceptive trait, it should be understood that it is fully within the scope of the present disclosure to use the same method and computer program implementing the method to decrease the value of one or more perceptive traits.

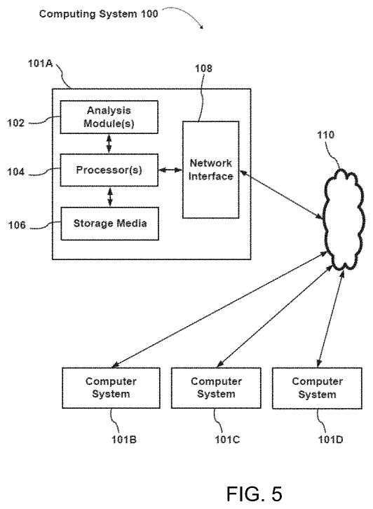

FIG. 5 shows an example embodiment of a computing system 100 that may be used to perform the actions explained with reference to FIGS. 1 through 4. Any form of storage medium, readable by various parts of the computer system 100, may comprise a computer program having logic operable to cause the computing system 100 or parts thereof to perform the actions as explained with reference to FIGS. 1 through 4. The computing system 100 may be an individual computer system 101A or an arrangement of distributed computer systems. The individual computer system 101A may include one or more analysis modules 102 that may be configured to perform various tasks according to some embodiments, such as the tasks explained with reference to FIG. 5. To perform these various tasks, the analysis module 102 may operate independently or in coordination with one or more processors 104, which may be connected to one or more storage media 106. A display device such as a graphic user interface of any known type may be in signal communication with the processor 104 to enable user entry of commands and/or data and to display results of execution of a set of instructions according to the present disclosure.

The processor(s) 104 may also be connected to a network interface 108 to allow the individual computer system 101A to communicate over a data network 110 with one or more additional individual computer systems and/or computing systems, such as 101B, 101C, and/or 101D (note that computer systems 101B, 101C and/or 101D may or may not share the same architecture as computer system 101A, and may be located in different physical locations, for example, computer systems 101A and 101B may be at a well drilling location, while in communication with one or more computer systems such as 101C and/or 101D that may be located in one or more data centers, and/or located in varying countries on different continents).

A processor may include, without limitation, a microprocessor, microcontroller, processor module or subsystem, programmable integrated circuit, programmable gate array, or another control or computing device.

The storage media 106 may be implemented as one or more computer-readable or machine-readable storage media, e.g., non-transitory storage media. The storage media may comprise logic operable to cause the computer system to perform the actions described above with reference to FIGS. 1 through 4. Note that while in the example embodiment of FIG. 5 the storage media 106 are shown as being disposed within the individual computer system 101A, in some embodiments, the storage media 106 may be distributed within and/or across multiple internal and/or external enclosures of the individual computing system 101A and/or additional computing systems, e.g., 101B, 101C, 101D. Storage media 106 may include, without limitation, one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories; magnetic disks such as fixed, floppy and removable disks; other magnetic media including tape; optical media such as compact disks (CDs) or digital video disks (DVDs); or other types of storage devices. Note that computer instructions to cause any individual computer system or a computing system to perform the tasks described above may be provided on one computer-readable or machine-readable storage medium, or may be provided on multiple computer-readable or machine-readable storage media distributed in a multiple component computing system having one or more nodes. Such computer-readable or machine-readable storage medium or media may be considered to be part of an article (or article of manufacture). An article or article of manufacture can refer to any manufactured single component or multiple components. The storage medium or media can be located either in the machine running the machine-readable instructions, or located at a remote site from which machine-readable instructions can be downloaded over a network for execution.

It should be appreciated that computing system 100 is only one example of a computing system, and that any other embodiment of a computing system may have more or fewer components than shown, may combine additional components not shown in the example embodiment of FIG. 5, and/or the computing system 100 may have a different configuration or arrangement of the components shown in FIG. 5. The various components shown in FIG. 5 may be implemented in hardware, software, or a combination of both hardware and software, including one or more signal processing and/or application specific integrated circuits.

Further, the acts of the processing methods described above may be implemented by running one or more functional modules in information processing apparatus such as general purpose processors or application specific chips, such as ASICs, FPGAs, PLDs, or other appropriate devices. These modules, combinations of these modules, and/or their combination with general hardware are all included within the scope of the present disclosure.

In light of the principles and example embodiments described and illustrated herein, it will be recognized that the example embodiments can be modified in arrangement and detail without departing from such principles. The foregoing discussion has focused on specific embodiments, but other configurations are also contemplated. In particular, even though expressions such as in “an embodiment,” or the like are used herein, these phrases are meant to generally reference embodiment possibilities, and are not intended to limit the disclosure to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise. Although only a few examples have been described in detail above, those skilled in the art will readily appreciate that many modifications are possible within the scope of the described examples. Accordingly, all such modifications are intended to be included within the scope of this disclosure as defined in the following claims.

What is claimed is:

1. A method for generating an image of a human face expected to have at least one changed perceptive trait, comprising:
encoding an image of a human face into a vector to generate an input image vector;
applying a scaled direction vector to the input image vector to generate a modified input image vector; and
converting the modified input image vector into a morphed image of the actual human face;
wherein the scaled direction vector is determined using an artificial neural network trained to associate human faces to with ratings of perceptive traits to determine a unit direction vector, the unit direction vector based on at least one chosen perceptive trait, an amplitude for scaling the unit direction vector determined by a limit on change in facial features obtainable by known treatments.

2. The method of claim 1 wherein the unit direction vector is determined by selecting, from among a plurality of human face rating images, ones of the human face rating images rated highest and ones of the human face rating images rated lowest, the rating performed by the artificial neural network for the at least one chosen perceptive trait, and determining a direction of the unit direction vector from vector representations of the selected highest-rated images and lowest-rated images.

3. The method of claim 2 wherein the human face rating images comprise randomly generated artificial human face images, the randomly generated artificial human face images generated by a generative adversarial network.

4. The method of claim 1 further comprising using the input image and the morphed image to automatically generate at least one recommended treatment to cause the human face from which the input image was made to most closely match the morphed image.

5. The method of claim 4 wherein the automatically generating the at least one recommended treatment comprises:
entering the input image and the morphed image into an artificial neural network trained for change detection to detect changed specific regions in the input image;
comparing the detected changed regions to face region masks known to be affected by specific treatments; and
if an intersection of at least one of the detected changed regions with at least one of the face region masks is greater than a predefined threshold, the specific treatment is added to a suggested treatment list.

6. The method of claim 5 wherein the changed regions are detected by calculating a difference image between the image of an actual human face and the morphed image.

7. The method of claim 5 wherein the changed regions are detected by entering the image of an actual human face and the morphed image into an artificial neural network trained on change detection.

8. The method of claim 4 wherein the automatically generating the at least one recommended treatment comprises entering the input image and the morphed image into an artificial neural network trained by entering before treatment images and after treatment images of actual human faces having undergone at least one treatment.

9. The method of claim 4 wherein the automatically generating the at least one recommended treatment comprises comparing a direction vector of the input image with reference to the morphed image with direction vectors of each of a plurality of treatments and selecting one of the plurality of treatments that most closely matches the direction vector of the input image with reference to the morphed image.

10. The method of claim 1 wherein the encoded image is of an actual human face.

11. A computer program stored in a non-transitory computer readable medium, the program comprising logic operable to cause a programmable computer to perform acts comprising:
encoding an image of a human face into a vector to generate an input image vector;
applying a scaled direction vector to the input image vector to generate a modified input image vector; and
converting the modified input image vector into a morphed image of the actual human face;
wherein the scaled direction vector is determined by training a first artificial neural network to determine changes in appearance of human face images corresponding to changes in perceptive traits to determine a direction vector based on at least one chosen perceptive trait, and an amplitude for scaling the direction vector is determined by a limit on change in facial features obtainable by known treatments.

12. The computer program of claim 11 wherein the unit direction vector is determined by selecting, from among a plurality of human face rating images, ones of the human face rating images rated highest and ones of the human face rating images rated lowest, the rating performed by the artificial neural network for the at least one chosen perceptive trait, and determining a direction of the unit direction vector from vector representations of the selected highest-rated images and lowest-rated images.

13. The computer program of claim 12 wherein the human face rating images comprise randomly generated artificial human face images, the randomly generated artificial human face images generated by a generative adversarial network.

14. The computer program of claim 11 further comprising using the input image and the morphed image to automatically generate at least one recommended treatment to cause the human face from which the input image was made to most closely match the morphed image.

15. The computer program of claim 14 wherein the automatically generating the at least one recommended treatment comprises:
entering the input image and the morphed image into an artificial neural network trained for change detection to detect changed specific regions in the input image;
comparing the detected changed regions to face region masks known to be affected by specific treatments; and
if an intersection of at least one of the detected changed regions with at least one of the face region masks is greater than a predefined threshold, the specific treatment is added to a suggested treatment list.

16. The computer program of claim 15 wherein the changed regions are detected by calculating a difference image between the image of an actual human face and the morphed image.

17. The computer program of claim 15 wherein the changed regions are detected by entering the image of an actual human face and the morphed image into an artificial neural network trained on change detection.

18. The computer program of claim 11 wherein the automatically generating the at least one recommended treatment comprises entering the input image and the morphed image into an artificial neural network trained by entering before treatment images and after treatment images of actual human faces having undergone at least one treatment.

19. The computer program of claim 11 wherein the automatically generating the at least one recommended treatment comprises comparing a direction vector of the input image with reference to the morphed image with direction vectors of each of a plurality of treatments and selecting one of the plurality of treatments that most closely matches the direction vector of the input image with reference to the morphed image.

20. The computer program of claim 1 wherein the encoded is of an actual human face.
